(19) United States
(12) Patent Application Publication
(10) Pub. No.: US 2023/0263746 A1
(43) Pub. Date: Aug. 24, 2023

(54) Synergistic Polyamine Combinations And Methods Therefor

(71) Applicant: Compound Solutions Inc., Carlsbad, CA (US)

(72) Inventor: Matthew Titlow, Carlsbad, CA (US)

(73) Assignee: Compound Solutions Inc., Carlsbad, CA (US)

(21) Appl. No.: 18/173,321

(22) Filed: Feb. 23, 2023

Related U.S. Application Data
(60) Provisional application No. 63/356,584, filed on Jun. 29, 2022.
(60) Provisional application No. 63/312,934, filed on Feb. 23, 2022.

(51) Int. Cl. A61K 31/132; A61K 9/00

(52) U.S. Cl. CPC A61K 31/132; A61K 9/0056; A61K 9/0014

(57) ABSTRACT

Compositions and methods are provided to increase the healthspan of a subject. Most typically, contemplated compositions are formulated for oral administration and comprise a synergistic combination of a first and a second polyamine (e.g., spermine and spermidine).

This application claims priority to our copending US Provisional Patent applications with the Ser. No. 63/312,934, filed Feb. 23, 2022, and Ser. No. 63/356,584, filed Jun. 29, 2022, both of which are incorporated by reference herein.

### FIELD OF THE INVENTION

The field of the invention is compositions and methods for metabolic modulation using a plurality of chemically distinct polyamines, and especially as it relates to synergistic effects of two or more polyamines.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Polyamines such as spermidine, spermine, and putrescine are commonly found in a variety of food items ranging from breast milk to edible plants. In addition, other polyamines such as putrescine may also be produced by microbial decarboxylase activity during spoilage or fermentation of plant foods. While common throughout a large spectrum of comestibles, the content and profile of polyamines varies greatly from one food type to another. For example, among plant-derived foods, cereals, legumes, and soybeans have the highest content of spermidine and spermine, while putrescine is often found at significant levels in in vegetables and fruits, and particularly citrus fruits.

Spermidine appears to be the predominant polyamine in food items. The food categories with the highest contents of spermidine and spermine are cereals, legumes, and soy derivatives. For example, wheat germ and soybeans provide spermidine at 2,437 nmol/g and 1,425 nmol/g, spermine at 722 nmol/g and 341 nmol/g, respectively. Mushrooms, peas, hazelnuts, pistachios, spinach, broccoli, cauliflower, and green beans also contain significant amounts of both polyamines, with the lowest levels in fruit. In contrast, putrescine is found in virtually all foods of plant origin, and is particularly abundant in fruits and vegetables, notably citrus fruits (1,554 nmol/g) and green peppers (794 nmol/g). There are also high amounts of putrescine in wheat germ (705 nmol/g) and soybean sprouts (507 nmol/g). See e.g., Food. Front. Nutr. 6:108 for a review of polyamines in food.

Polyamines have typically been associated with a high antioxidant activity and have therefore been reported as being potentially beneficial in aging, cardiovascular disease, chronic inflammatory pathologies, DNA repair, and metabolic disorders. For example, spermidine has been reported among other activities as a physiological autophagy inducer acting as an anti-aging vitamin in humans (see e.g., Autophagy 2019, VOL. 15, NO. 1, 165-168), and to promote human hair growth and to modulate human epithelial stem cell functions (see e.g., PLoS One July 2011, Vol. 6, Issue 7, e22564). These advantages have inspired formulation of nutritional supplements enriched in spermidine, typically from enriched wheat germ. However, and despite the relatively large variety of apparent biological effects, the exact pleiotropic mechanism of action of spermidine (and polyamines in general) has remained elusive. Additionally, there is a significant lack of recognition if and how multiple and distinct polyamine compounds interact in physiological context.

More recently, as discussed in WO 2010/081204, it was discovered that selected polyamine compounds can induce deacetylation or can maintain a condition of hypoacetylation of histone H3, can activate autophagy, and can be used in a treatment to activate or reinstate a normal autophagic pathway, which may contribute to a beneficial effect on the protection of coronary heart disease and atherosclerosis. WO 2010/081204 refers to extending the healthy lifespan of a subject using a polyamine compound that must be present in an amount that activates the cellular autophagic pathway. However, it has been shown that at least some of the polyamine compounds, when used individually, did not extend the healthspan of subjects.

Thus, even though various composition and methods for polyamines are known in the art, all or almost all of them suffer from several drawbacks. Therefore, there remains a need for improved composition and methods for polyamines in which two or more polyamines synergize with respect to one or more physiological parameters, especially as it relates to increasing healthspan.

### SUMMARY OF THE INVENTION

The inventor has discovered various nutritionally and/or pharmaceutically acceptable compositions and methods comprising at least two chemically distinct polyamines in synergistic quantities with respect to increasing healthspan.

Therefore, the inventor contemplates a nutritional composition that includes a synergistic combination of a first and a second polyamine in a mixture with a nutritionally acceptable carrier in which the first and second polyamine are present in quantities that synergistically increase a healthspan of a subject. Most preferably, the composition is formulated for oral consumption or topical application.

For example, the first and second polyamines are chemically distinct and can be selected from spermidine, spermine, putrescine, cadaverine, agmatine, and ornithine, each of which may be present in the composition as a free base or as a hydrochloride (or other) salt or be provided from a natural source. Among other options, a dosage unit of contemplated compositions may contain spermidine in an amount of at least 0.1 mg, spermine in an amount of at least 0.05 mg, putrescine in an amount of at least 0.01 mg, cadaverine in an amount of at least 0.1 mcg, agmatine in an amount of at least 1 mg, and/or L-ornithine in an amount of at least 0.01 mg.

Viewed from a different perspective, a dosage unit of the composition may therefore contain spermidine in an amount of equal or less than 50 mg, spermine in an amount of equal or less than 20 mg, putrescine in an amount of equal or less than 5 mg, cadaverine in an amount of equal or less than 50 mcg, agmatine in an amount of equal or less than 1,000 mg, and/or L-ornithine in an amount of equal or less than 1,000 mg.

In some embodiments, the first and second polyamine are present in synergistic quantities with respect to increasing the healthspan of a subject by increasing locomotor activity.

In further embodiments, the first and second polyamine are present in synergistic quantities with respect to increasing the healthspan of a subject by preserving or increasing the health of thin or thick filaments of cellular muscle fibers.

In yet further embodiments, the first and second polyamine are present in synergistic quantities with respect to increasing the healthspan of a subject by preserving or increasing the stimulation of regions of the brain associated with reward or happiness.

Alternatively, or additionally, the first and second polyamine are present in synergistic quantities with respect to increasing the healthspan of a subject by reducing cortisol-mediated effects in the nervous system.

In additional embodiments, the first and second polyamine may also be present in synergistic quantities with respect to increasing the healthspan of a subject by preserving or increasing the energy yield of metabolic processes, and/or in synergistic quantities with respect to increasing the healthspan of a subject by preserving or increasing the health of extracellular matrices.

Moreover, it is contemplated that the first and second polyamine are present in synergistic quantities with respect to increasing the healthspan of a subject by preserving or increasing intracellular NAD⁺ or ATP concentration.

The first and second polyamine are also contemplated to increase the healthspan of a subject by preserving or increasing mitochondrial biogenesis when the first and second polyamine are present in synergistic quantities.

Further, it is contemplated that the first and second polyamine are present in synergistic quantities with respect to increasing the healthspan of a subject by preserving or increasing glucose uptake into cells.

As will be readily appreciated, contemplated compositions may also comprise one or more additional components that will have beneficial effects on a subject's metabolism. Additional components that support metabolism are contemplated to include ingredients like fish oil, melatonin, vitamins, capsaicin, theacrine, methylliberine, berberine, healthy acids (e.g., dietary mono- and/or dicarboxylic acids), turmeric, caffeine, green tea, protein, or fiber.

Contemplated compositions may be formulated as a capsule, tablet, gummy, chew, gel, powder, drink, or a cosmetic.

In another aspect of the inventive subject matter, the inventors contemplate a method of modulating a healthspan of a subject that includes a step of orally or topically administering a nutritional composition comprising a synergistic combination of a first and second polyamine that are present in synergistic quantities with respect to increasing the healthspan of a subject.

Possible examples of the first and second polyamine, which are chemically distinct in the administered composition, include spermidine, spermine, putrescine, cadaverine, agmatine, and ornithine, which may be present in the composition as a free base or as a hydrochloride (or other) salt or be provided from a natural source. In preferred embodiments, the first and/or second polyamine are present in the composition in synergistic quantities with respect to the healthspan.

Among other options, the method of administering the composition may be such that the dosage unit of the composition contains spermidine in an amount of between 0.1 mg and 50 mg, spermine in an amount of between 0.05 mg and 20 mg, putrescine in an amount of between 0.01 mg and 5 mg, cadaverine in an amount of between 0.1 mcg and 50 mcg, agmatine in an amount of between 1 mg and 1,000 mg, and/or L-ornithine in an amount of between 0.01 mg and 1 g.

In additional options, the method of administering the composition may modulate a subject's healthspan by preserving or increasing locomotor activity, health of thin or thick filaments of cellular muscle fibers, stimulation of brain regions associated with reward or happiness, energy yield of metabolic processes, health of extracellular matrices, intracellular concentration of NAD⁺ or ATP, mitochondrial biogenesis, and/or glucose uptake into cells, and/or decreases concentration of cortisol in the circulatory and/or nervous system.

In some embodiments, the method of administering the composition may be such that the composition includes one or more of the following additional ingredients: fish oil, melatonin, vitamins, capsaicin, theacrine, methylliberine, berberine, healthy acids, turmeric, caffeine, green tea, protein, and/or fiber.

Moreover, it is contemplated that the method of administering the composition to modulate a subject's healthspan is performed for research purposes. It is further contemplated that the method of administering the composition to modulate a subject's healthspan could include research subjects selected from the following group: Nematoda, Nematomorpha, Drosophilidae, Cyprinidae, Rodentia, and/or Choanozoa.

In yet another aspect of the inventive subject matter, the inventors contemplate a method of manufacturing a nutritional composition, which includes a step of compounding a first and a second polyamine with a nutritionally acceptable carrier. Typically, this method includes a step of formulating the first and the second polyamine and the carrier into an oral or topical formulation.

The inventors further contemplate the first and second polyamine to be present in the formulation in quantities that synergistically increase the healthspan of a subject.

As with the other method contemplated by the inventors, possible examples of the first and second polyamine, which are chemically distinct when compounded for this method of manufacturing a nutritional composition, are spermidine, spermine, putrescine, cadaverine, agmatine, and/or ornithine, which may be present in the composition as a free base or as a hydrochloride (or other) salt or be provided from a natural source.

Among other options, the method of administering the composition may be such that a dosage unit of the composition contains spermidine in an amount of at least 0.1 mg, spermine in an amount of at least 0.05 mg, putrescine in an amount of at least 0.01 mg, cadaverine in an amount of at least 0.1 mcg, agmatine in an amount of at least 1 mg, and/or L-ornithine in an amount of at least 0.01 mg.

In an additional option contemplated by the inventors, the method of administering the composition may be such that a dosage unit of the composition contains spermidine in an amount of equal or less than 50 mg, spermine in an amount of equal or less than 20 mg, putrescine in an amount of equal or less than 5 mg, cadaverine in an amount of equal or less than 50 mcg, agmatine in an amount of equal or less than 1,000 mg, and/or L-ornithine in an amount of equal or less than 1,000 mg.

It is further contemplated by the inventors that the method of manufacturing the nutritional composition includes formulating the first and the second polyamine into an oral formulation that is a powdered formulation. This powdered formulation is additionally contemplated to be encapsulated or compressed to form a tablet or to form a single-dosage unit.

The inventors also contemplate that the method of manufacturing the nutritional composition includes formulating the first and the second polyamine into an oral formulation that is a ready-to-use beverage.

Moreover, the inventors also contemplate the method of manufacturing the nutritional composition to further comprise advertising or marketing the nutritional composition as related to healthspan.

Various objects, features, aspects, and advantages of the inventive subject matter will become more apparent from the following detailed description of preferred embodiments, along with the accompanying drawings.

### BRIEF DESCRIPTION OF THE DRAWINGS

FIG. 1 is a graph depicting exemplary results for lifespan in a C. elegans model using negative and positive (resveratrol) controls.

FIG. 2 is a graph depicting exemplary results for lifespan in a C. elegans model using negative and positive (resveratrol) controls, nicotinamide mononucleotide (NMN), spermidine, spermine, and a spermine/spermidine-combination.

FIG. 3 is a graph depicting exemplary results for lifespan in a C. elegans model using a negative control, nicotinamide mononucleotide (NMN), spermidine, spermine, and a spermine/spermidine-combination.

FIG. 4 is a graph depicting exemplary results for lifespan in a C. elegans model using a negative control, spermidine, spermine, and a spermine/spermidine-combination.

FIG. 5 is a table displaying the mean lifespan extension in the C. elegans model of FIGS. 1-4 when treated respectively with 100 μM resveratrol, 500 μM NMN, 20 μg/mL spermidine, 6 μg/mL spermine, and the spermine/spermidine-combination. In particular, the table displays the mean lifespan of each treated C. elegans model in the units of days, the percentage change of the mean lifespan when compared to control, and the p-value of the mean lifespan when compared to control.

FIG. 6 is a table displaying a pairwise comparison of mean lifespan in a C. elegans model of FIGS. 1-4 when treated respectively with 500 μM NMN, 20 μg/mL spermidine, 6 μg/mL spermine, and the spermine/spermidine-combination. In addition to displaying the p-value of each treatment when compared to control, the table displays the p-value of each treatment when compared to NMN, spermidine, and spermine.

FIG. 7 depicts a method of scoring locomotor activity in C. elegans wherein the animal's displacement from a fixed boundary determines an activity score. The activity scores range from 0<s≤0.1 (sedentary), to 0.1<s≤0.4 (moderately active), to 0.4<s≤1 (highly active).

FIG. 8 is a graph depicting exemplary results for the locomotor activity of a wildtype C. elegans model with respect to age. The locomotor activity of the C. elegans model appears to decline with age.

FIG. 9 is a graph depicting exemplary results for the locomotor activity of a wildtype C. elegans model and a resveratrol-treated C. elegans model with respect to age. As the C. elegans move into late life on days 10 and 12, the resveratrol-treated C. elegans remain more active than the wildtype C. elegans.

FIG. 10 is a graph depicting exemplary results for the locomotor activity of a wildtype C. elegans model and an NMN-treated C. elegans model with respect to age. As the C. elegans move into late life on days 10 and 12, the NMN-treated C. elegans show significantly higher levels of activity than the wildtype C. elegans.

FIG. 11 is a graph depicting exemplary results for the locomotor activity of a wildtype C. elegans model and a spermidine-treated C. elegans model with respect to age. C. elegans treated with 20 μg/mL spermidine do not show statistically significant difference in activity levels compared to wildtype C. elegans.

FIG. 12 is a graph depicting exemplary results for the locomotor activity of a wildtype C. elegans model and a spermine-treated C. elegans model with respect to age. C. elegans treated with 6 μg/mL spermine show significantly more activity on day 8, but this effect does not seem to persist significantly into later ages.

FIG. 13 is a graph depicting exemplary results for the locomotor activity of a wildtype C. elegans model and a C. elegans model treated with a spermine/spermidine-combination with respect to age. C. elegans treated with 20 μg/mL spermidine and 6 μg/mL spermine in combination show significantly more activity than the wildtype C. elegans on day 12.

FIG. 14 is a graph depicting exemplary results for the locomotor activity of C. elegans respectively treated with spermidine, spermine, and the spermine/spermidine-combination.

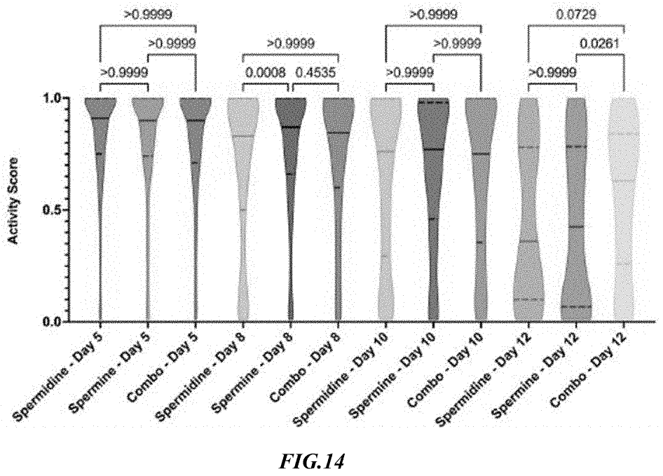

### DETAILED DESCRIPTION

The inventor has now discovered that compositions comprising at least two chemically distinct polyamines can advantageously, and in certain embodiments synergistically, modulate one or more physiological parameters associated with healthspan. In addition, contemplated compositions have also demonstrated reduced mortality rates and increase in overall lifespan, particularly in the second half of life as seen in a C. elegans model.

As used herein, the term “healthspan” refers to healthy aging. Healthspan, or healthy aging, can be observed and quantified through a variety of systemic phenomena or metrics, and especially locomotor activity, which in turn is influenced by of one or more underlying physiological processes or conditions that include health state and/or functioning of thin or thick filaments of cellular muscle fibers, stimulation of brain regions associated with reward or happiness, energy yield of metabolic processes, health state and/or functioning of extracellular matrices, intracellular concentration of NAD+ or ATP, mitochondrial biogenesis, glucose uptake into cells, and concentration of cortisol in the circulatory and/or nervous system. Further examples associated with healthspan or healthy aging include longevity, gut health, intestinal barrier integrity, skin health, dermal barrier integrity, cognition, memory, and/or energy metabolism.

In this context, it should be appreciated that healthspan is to be distinguished from survivability, lifespan, and life expectancy. Survivability, lifespan, and life expectancy depend upon a quantifiable amount of time that a subject may live. In contrast, healthspan depends upon the status of physiological parameters that affect a subject's health as the subject ages. Therefore, a subject can potentially have a relatively strong survivability and long lifespan, yet have a poor healthspan (for example, by having weak thin and thick filaments in muscle fibers, poor glucose uptake into cells, deficient mitochondrial biogenesis, and so on). In especially advantageous aspects, and as shown in more detail below, contemplated compositions and methods delay a decline in normal locomotor activity that would otherwise be observed in untreated subjects. Such delay of decline was especially pronounced in the latter part of the subject's lifespan (e.g., latter half, latter third, latter quarter, etc.), which may or may not be accompanied by an increase in lifespan.

With respect to exemplary compositions that increase healthspan of a subject to which the composition is administered, a composition may be formulated as a nutritional composition for oral administration as a capsule representing a single dosage unit. In such example, the capsule will typically deliver a total of about 3 mg spermidine, 0.5 mg spermine, and optionally 100 mg agmatine and/or 0.5 mg putrescine, typically in association with nutritionally acceptable excipients. Moreover, it is contemplated that in some embodiments a single dosage unit will have a total weight of equal or less than 1,000 mg, equal or less than 750 mg, and more typically equal or less than 500 mg, equal or less than 250 mg. As such, the single dosage unit will contain at least 10 wt %, or at least 20 wt %, or at least 50 wt %, or at least 70 wt %, or at least 85 wt % of at least two chemically distinct polyamines.

With respect to the particular choice of the specific chemically distinct polyamines it should be appreciated that all known and nutritionally acceptable polyamines are deemed suitable for use herein, and particularly preferred polyamines will include spermidine, spermine, putrescine, cadaverine, agmatine, and ornithine. As will be readily appreciated, the polyamines may be prepared or isolated from various plant materials or may be provided as synthetic compounds. Where the polyamine(s) is/are synthetic compounds, it is particularly preferred that the polyamines are provided in form of their free base or as a hydrochloride salt. Advantageously, such forms have significant improved stability and can be readily dispensed in measured quantities. An exemplary method of preparing such free base and salt forms is disclosed in our co-pending US Provisional Patent application with Ser. No. 18/085,157, which was filed Dec. 20, 2022, and which is incorporated by reference in its entirety herein. Alternatively, one or more of the polyamines may also be obtained from a commercial source or provided in form of a plant material or plant extract that is at least partially enriched in the polyamine(s).

Therefore, the purity of the individual polyamine may vary considerably, and it is contemplated that the purity of one or more of the polyamines (by wt%) will be at least 0.5%, or at least 1.0%, or at least 5.0%, or at least 10%, or at least 20%, or at least 30%, or at least 40%, or at least 50%, or at least 60%, or at least 70%, or at least 80%, or at least 90%, or at least 95%, or at least 97%, or even higher. Most commonly, where the purity is at or below 95%, at or below 90%, at or below 85%, at or below 80%, it is contemplated that the remaining material in the polyamine preparation will be of plant or milk origin.

With respect to suitable quantities of the polyamines, it is generally preferred that the polyamines are present in quantities effective to exert a beneficial (and most preferably a synergistic) effect on at least one physiological parameter, and especially on one or more of locomotor activity, health of thin or thick filaments of cellular muscle fibers, stimulation of brain regions associated with reward or happiness, energy yield of metabolic processes, health of extracellular matrices, intracellular concentration of NAD+or ATP, mitochondrial biogenesis, glucose uptake into cells, concentration of cortisol in the nervous system, autophagy, longevity, gut barrier integrity, dermal barrier integrity, cognition, glucose metabolism, and lipid metabolism as described in more detail below. Therefore, a dosage unit may be prepared that provides selected quantities of the polyamines as noted below. In that context it should be noted that a dosage unit may be formulated as a tablet, capsule, or other solid dosage form, but also as a liquid such as a ready to use (typically single serving) drink. In further contemplated aspects, the single dosage unit may also be defined as a specific quantity of a liquid or solid bulk preparation such as a recommended daily intake (e.g., expressed in specific weight or other measuring unit).

For example, spermidine may be present in a dosage unit in an amount of at least 0.1 mg, or at least 0.5 mg, or at least 0.75 mg, or at least 1 mg, or at least 2 mg, or at least 3 mg, or at least 4 mg, or at least 5 mg, or at least 6 mg, or at least 7 mg, or at least 8 mg, or at least 9 mg, or at least 10 mg, or at least 15 mg, or at least 20 mg, or at least 25 mg, or at least 30 mg, or at least 35 mg, or at least 40 mg, or at least 50 mg, and even more. Thus, suitable dosage ranges include between 0.1 mg and 1 mg, between 1 mg and 10 mg, between 10 mg and 25 mg, between 25 mg and 50 mg, and even more.

In another example, spermine may be present in a dosage unit in an amount of at least 0.01 mg, or at least 0.05 mg, or at least 0.07 mg, or at least 0.1 mg, or at least 0.2 mg, or at least 0.4 mg, or at least 0.6 mg, or at least 0.8 mg, or at least 1 mg, or at least 2 mg, or at least 3 mg, or at least 4 mg, or at least 5 mg, or at least 6 mg, or at least 7 mg, or at least 8 mg, or at least 9 mg, or at least 10 mg, or at least 15 mg, or at least 20 mg, and even more. Thus, suitable dosage ranges include between 0.01 mg and 0.5 mg, between 0.5 mg and 5 mg, between 5 mg and 10 mg, between 10 mg and 20 mg, and even more.

In a further example, putrescine may be present in a dosage unit in an amount of at least 0.01 mg, or at least 0.05 mg, or at least 0.07 mg, or at least 0.1 mg, or at least 0.2 mg, or at least 0.3 mg, or at least 0.4 mg, or at least 0.5 mg, or at least 0.6 mg, or at least 0.7 mg, or at least 0.8 mg, or at least 0.9 mg, or at least 1 mg, or at least 1.5 mg, or at least 2.0 mg, or at least 2.5 mg, or at least 3.0 mg, or at least 3.5 mg, or at least 4.0 mg, or at least 5.0 mg, and even more. Thus, suitable dosage ranges include between 0.01 mg and 0.1 mg, between 0.1 mg and 1.0 mg, between 1.0 mg and 2.5 mg, between 2.5 mg and 5.0 mg, and even more.

In still another example, cadaverine may be present in a dosage unit in an amount of at least 0.1 mcg, or at least 0.5 mcg, or at least 0.75 mcg, or at least 1 mcg, or at least 2 mcg, or at least 3 mcg, or at least 4 mcg, or at least 5 mcg, or at least 6 mcg, or at least 7 mcg, or at least 8 mcg, or at least 9 mcg, or at least 10 mcg, or at least 15 mcg, or at least 20 mcg, or at least 25 mcg, or at least 30 mcg, or at least 35 mcg, or at least 40 mcg, or at least 50 mcg, and even more. Thus, suitable dosage ranges include between 0.1 mcg and 1 mcg, between 1 mcg and 10 mcg, between 10 mcg and 25 mcg, between 25 mcg and 50 mcg, and even more.

In a further example, agmatine may be present in a dosage unit in an amount of at least 1.0 mg, or at least 5.0 mg, or at least 7.5 mg, or at least 10 mg, or at least 20 mg, or at least 30 mg, or at least 40 mg, or at least 50 mg, or at least 60 mg, or at least 70 mg, or at least 80 mg, or at least 90 mg, or at least 100 mg, or at least 150 mg, or at least 200 mg, or at least 250 mg, or at least 300 mg, or at least 350 mg, or at least 400 mg, or at least 500 mg, or at least 750 mg, or at least 1,000 mg, and even more. Thus, suitable dosage ranges include between 1 mg and 100 mg, between 100 mg and 300 mg, between 300 mg and 600 mg, between 600 mg and 1,000 mg, and even more.

In yet a further example, low quantities of ornithine may be present in a dosage unit in an amount of at least 0.01 mg, or at least 0.05 mg, or at least 0.07 mg, or at least 0.1 mg, or at least 0.2 mg, or at least 0.3 mg, or at least 0.4 mg, or at least 0.5 mg, or at least 0.6 mg, or at least 0.7 mg, or at least 0.8 mg, or at least 0.9 mg, or at least 1 mg, or at least 1.5 mg, or at least 2.0 mg, or at least 2.5 mg, or at least 3.0 mg, or at least 3.5 mg, or at least 4.0 mg, or at least 5.0 mg, and even more. Thus, suitable dosage ranges include between 0.01 mg and 0.05 mg, between 0.05 mg and 0.1 mg, between 0.1 mg and 0.5 mg, between 0.5 mg and 1.0 mg, and even more. On the other hand, and especially in exercise related uses, ornithine may also be present in a dosage unit in an amount of at least 10 mg, or at least 50 mg, or at least 100 mg, or at least 300 mg, or at least 600 mg, or at least 1,000 mg, and even higher.

Therefore, a dosage unit of the composition may contain spermidine in an amount of at least 0.1 mg, spermine in an amount of at least 0.05 mg, putrescine in an amount of at least 0.01 mg, cadaverine in an amount of at least 0.1 mcg, agmatine in an amount of at least 1 mg, and/or L-ornithine in an amount of at least 0.01 mg. Viewed from a different perspective, a dosage unit of the composition may contain spermidine in an amount of equal or less than 50 mg, spermine in an amount of equal or less than 20 mg, putrescine in an amount of equal or less than 5 mg, cadaverine in an amount of equal or less than 50 mcg, agmatine in an amount of equal or less than 1,000 mg, and/or L-ornithine in an amount of equal or less than 1 mg, and/or a dosage unit of the composition may contain spermidine in an amount of between 0.1 mg and 50 mg, spermine in an amount of between 0.05 mg and 20 mg, putrescine in an amount of between 0.01 mg and 5 mg, cadaverine in an amount of between 0.1 mcg and 50 mcg, agmatine in an amount of between 1.0 mg and 1,000 mg, and/or L-ornithine in an amount of between 0.01 mg and 1 mg.

With respect to the synergistic ratios of the first to the second polyamine it is contemplated that the two polyamines may be present at a ratio (in weight %) of first to second polyamine (e.g., spermidine to spermine) of about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, or about 1:20. Viewed form a different perspective, contemplated synergistic rations may be found between 1:1 to 1:3, or between 1:3 to 1:6, or between 1:6 to 1:10, or between 3:1 to 1:1, or between 6:1 to 3:1, or between 10:1 to 6:1. In that regard, it should be noted that the strength of the synergistic effect may be an increase in the observed effect (relative to the effect of a single polyamine at the same dose) of at least 5%, or at least 10%, or at least 15%, or at least 20%, or at least 25%, and even higher. Still further, it should be noted that synergy may also be observed when the first and second polyamine are combined with a third polyamine, where first, second, and third polyamines are chemically distinct.

As will be readily appreciated, contemplated compositions may also comprise one or more additional components that will have beneficial effect on the intestinal tissue and/or microbiome of a subject ingesting the composition. For example, especially contemplated include various prebiotic compositions such as kombucha, human milk oligosaccharides, fructooligosaccharides (FOS), galactooligosaccharides (GOS), xylooligosaccharides (XOS), arabinoxylans (AX), 1-arabinose, various pectins, various gums, various polyphenols, beta glucans, inulin, short-chain fatty acids, medium chain triglycerides, tributyrin, beta-hydroxybutyrate, and acetoacetate.

Administration of contemplated compositions will be typically via oral route in one or more single dosage units per day, and/or in an amount effective to (preferably synergistically) modulate at least one physiological parameter of autophagy, longevity, gut barrier integrity, dermal barrier integrity, cognition, glucose metabolism, and lipid metabolism. As will be readily appreciated, such administration may beneficially improve signs and/or symptoms associated with one or more conditions such as (chronic and/or subacute) inflammation, degenerative disease, cardiovascular disease, (age-related) cognitive decline or impairment, intestinal dysfunction, metabolic dysfunction, obesity, and/or dermal barrier dysfunction.

In typical embodiments, administration of contemplated compositions will be daily using between 0.1 and 10, and most typically between 0.5 and 3 dosage units (e.g., 1) over a period of at least 1 week, or at least 2 weeks, or at least 1 month, or at least 2 months, or at least 3 months, or at least 6 months, or at least 1 year, and even longer. As will be readily appreciated, administration will preferably be daily, or at least twice weekly, or at least three times weekly. Alternatively, or additionally, administration is performed until at least one at least one physiological parameter is modulated as further discussed below.

As will be readily appreciated, contemplated compositions can be formulated in a wide variety of forms suitable for oral administration, including solid formulations and liquid formulations. For example, where the oral formulation is a solid formulation, contemplated compositions may be prepared as a bulk powder, a tablet, a capsules, a gummy, a lozenge, a dissolving film, a dragee, etc., or may be formulated as a snack or energy bar or other ready-to-eat food item. On the other hand, where the oral formulation is a liquid formulation, contemplated compositions may be prepared as a liquid concentrate, a ready-to-drink beverage, or an energy drink. Likewise, it should be appreciated that the compositions presented herein can also be formulated for dermal/topical use. Therefore, suitable formulations include creams, ointments, lotions, sera, soaps, shampoos/conditioners, etc.

### Examples

The following examples are provided to illustrate selected physiological markers and/or processes that can be beneficially, and in at least some cases synergistically modulated by the compositions presented herein. However, these examples are not intended to be limiting the inventive subject matter, and additional and/or alternative markers and processes are also expressly contemplated herein. Unless noted otherwise, measurement of these markers is known in the art and will follow well-known protocols.

One physiological marker found to be beneficially and synergistically modulated by the compositions presented herein is lifespan. In one example, the inventors placed wildtype age-synchronized C. elegans L1 larvae on agar plates seeded with live E. coli OP50 supplemented respectively with 100 μM resveratrol, 500 μM NMN, 20 μg/mL spermidine, 6 μg/mL, and a combination of 20 μg/mL spermidine with 6 μg/mL. An additional control group was given no treatment. All groups were loaded into chips at the L4-stage. From the 1st day of adulthood, the animals were switched to heat-killed OP50 plus the appropriate compound. Chips were scored daily until all animals were dead.

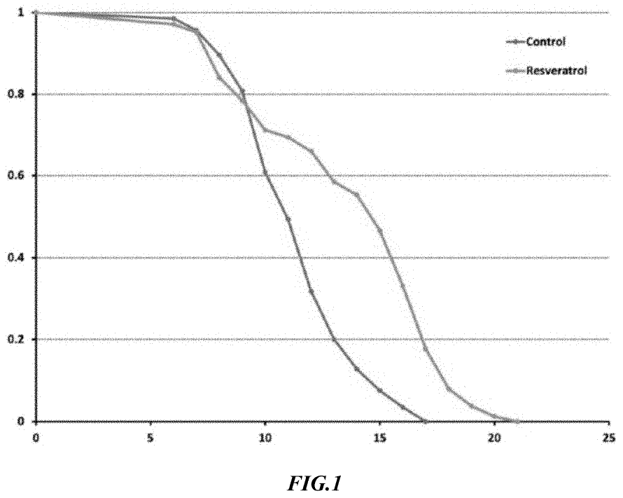

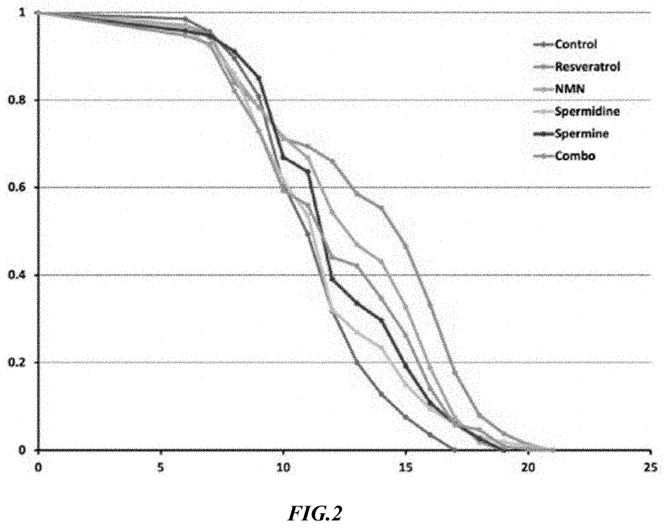

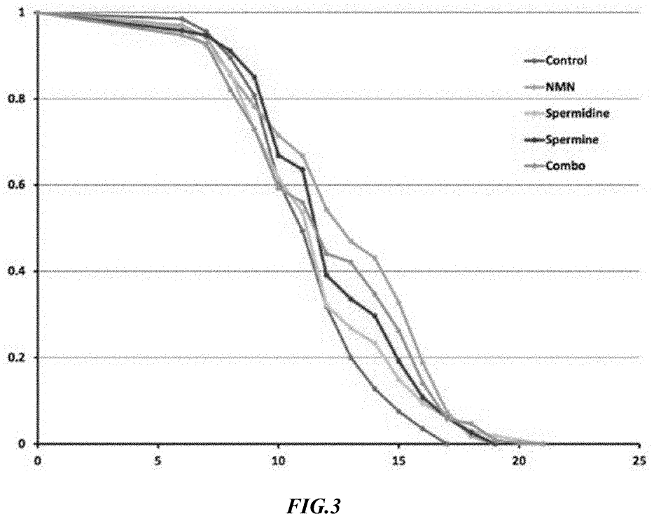

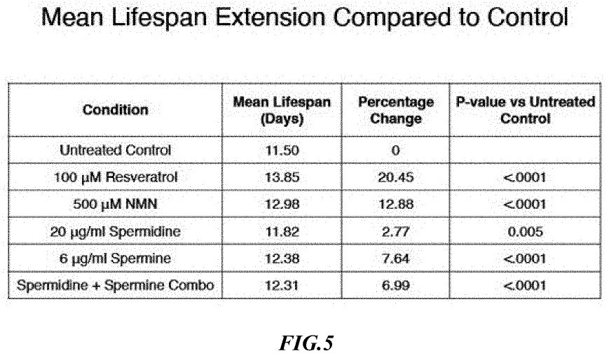

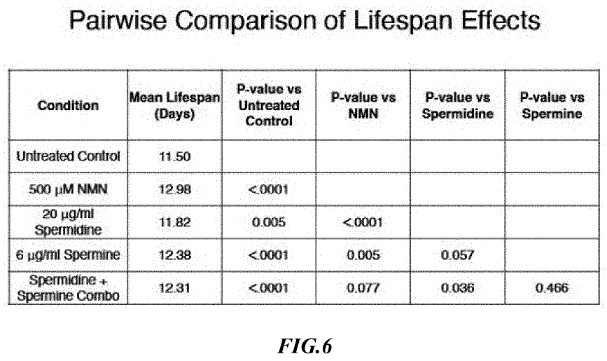

In this example, the groups treated with resveratrol and NMN showed the strongest increase to lifespan compared to control, as expected. The groups treated with spermidine alone showed the weakest effect on lifespan, yet still significant. The groups treated with spermine and the spermine/spermidine-combination had significant effects on lifespan. However, the effects of spermine vs the spermidine/spermine-combination treatment were different. See FIGS. 1-4 and the table in FIG. 5. The table in FIG. 6 provide statistical values for pairwise comparisons on lifespan with regard to different treatments.

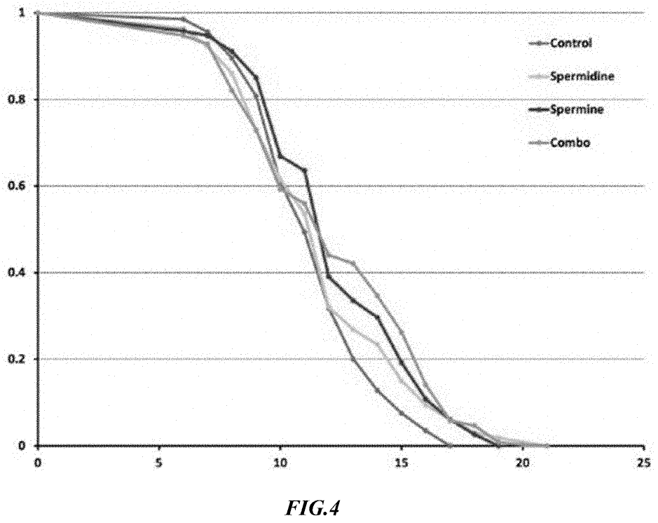

Notably, it should be appreciated that the differences between control and the treatments with spermine, spermidine, and the combination of spermine and spermidine were more pronounced in the second half or later portion in life. As can be seen from FIG. 4, the fraction of live organisms at the same date (and particularly at and after day 12, corresponding to the latter third in life) was increased over control in organisms treated with spermine and spermidine, and even further increased where the organisms were treated with the combination. As such, the mortality rate (on a population basis) was significantly reduced in the later portion of live.

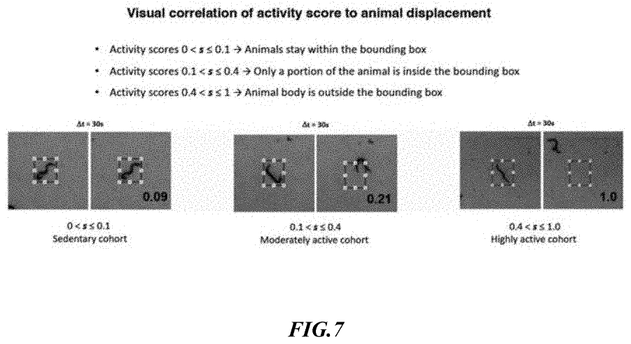

Upon further investigation, the inventor also discovered that healthspan presented an additional physiological marker that was beneficially and synergistically modulated by the compositions presented herein. In a set of experiments, healthspan was measured/quantified by observing the degree of locomotor activity an animal visually exhibits as it ages. Using C. elegans as test subjects, this measurement can be accomplished by assigning each animal a digitally fixed boundary and observing each animal's displacement from that boundary. See FIG. 7. In one example, the inventors placed wildtype age-synchronized C. elegans L1 larvae on agar plates seeded with live E. coli OP50 supplemented respectively with 100 μM resveratrol, 500 μM NMN, 20 μg/mL spermidine, 6 μg/mL, and a combination of 20 μg/mL spermidine with 6 μg/mL. An additional control group was given no treatment. All groups were loaded into chips at the L4-stage. From the 1st day of adulthood, the animals were switched to heat-killed OP50 plus the appropriate compound. The displacement of each animal from its assigned fixed boundary determined an activity score. The activity scores range from 0<s≤0.1 (sedentary), to 0.1<s≤0.4 (moderately active), to 0.4<s≤1 (highly active).

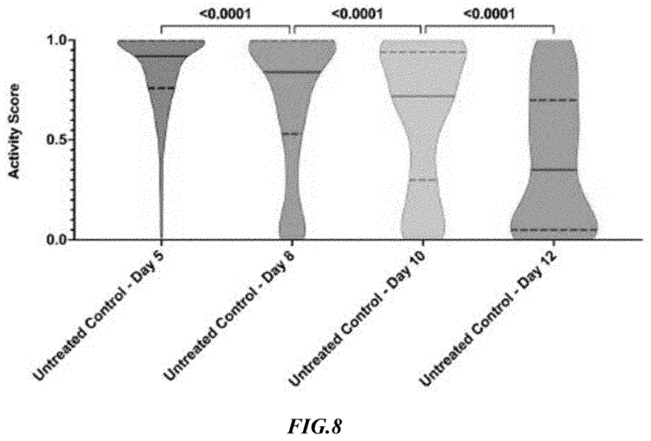

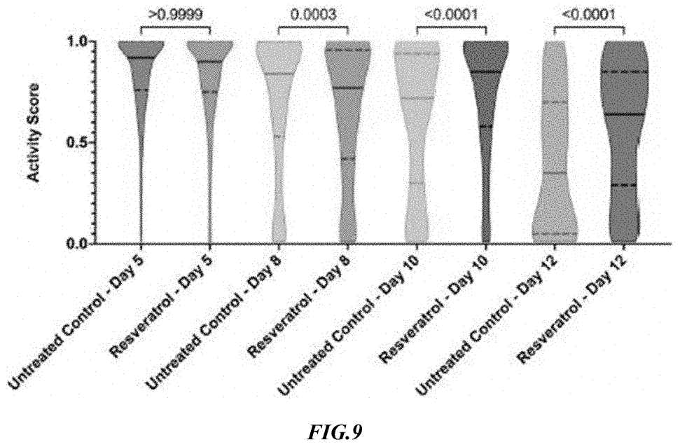

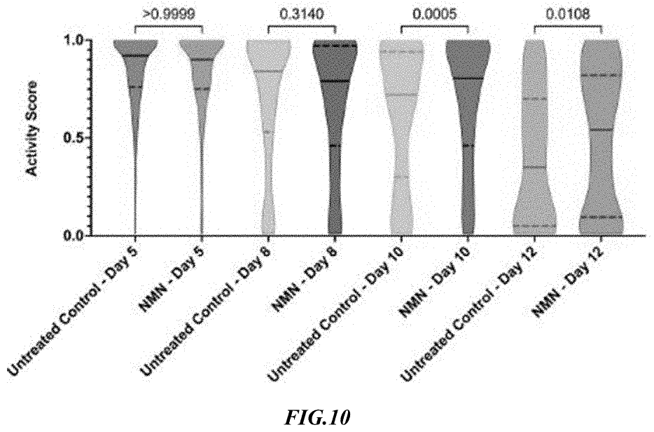

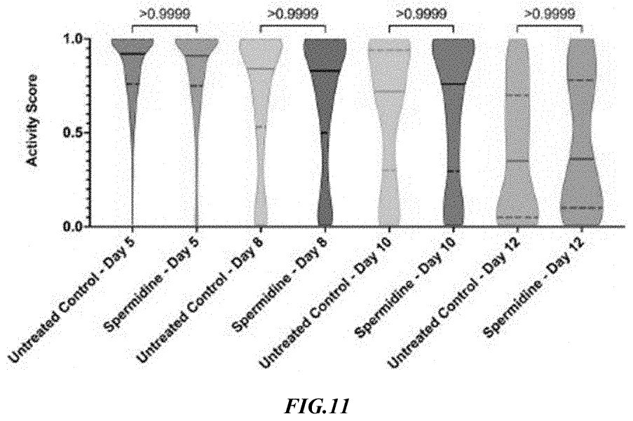

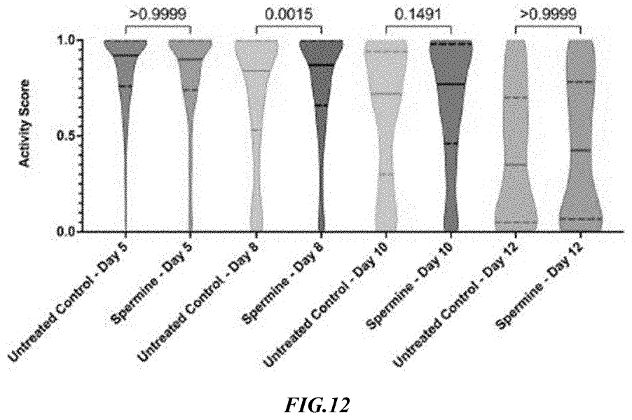

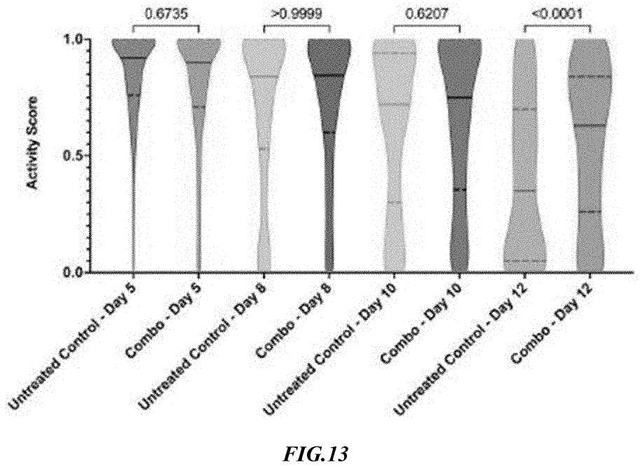

In this example, in the untreated control group, the majority of animals had a high activity score during early life, and the distribution became more skewed to low score as the animals became older. See FIG. 8. Groups treated with resveratrol and NMN showed significantly higher levels of activity than untreated groups by days 10 and 12 of animal life. See FIG. 9 and FIG. 10. The group treated with spermidine alone did not show any significant difference in activity levels compared to the untreated group even though spermidine treatment did lead to a slight lifespan extension compared to the untreated group. See FIG. 11. Notably, the group treated with spermine alone showed significant differences in activity levels compared to the untreated group on day 8 of animal life, but this effect did not persist into later ages. See FIG. 12. Remarkably, the group treated with the spermine/spermidine-combination showed significantly more activity than the untreated group on day 12 of animal life. See FIG. 13. This finding suggests that the effects of the combination treatment on activity may be acting via a different mechanism than that of lifespan extension. Viewed from a different perspective, and given that the effects on lifespan and healthspan were distinct as shown above, it should be appreciated that the beneficial effects on healthspan (and particularly in the later phases of life) are unexpected and could not have been predicted from increased lifespan data. This finding also suggests that spermine and spermidine have a beneficial and synergistic effect on the locomotor activity of animals as the animals age.

Based on the observed beneficial and synergistic effects on lifespan and healthspan, the inventor therefore also contemplates that numerous physiological parameters associated with increased lifespan and healthspan will be positively affected by the polyamine combinations presented herein. Among other suitable parameters, the inventor contemplates that the compositions presented herein will beneficially (and synergistically) modulate metabolism, the gut microbiome, autophagy, cognitive and/or memory function, gut barrier function, and/or dermal structure and skin barrier function, and that each of these beneficial effects can be quantitatively confirmed as discussed below.

Metabolism: Contemplated compositions can be administered to a healthy subject or a subject suffering from or diagnosed with a metabolic disorder such as dyslipidemia, loss of insulin sensitivity, prediabetes, type 2 diabetes, obesity, fatty liver (NASH). It should also be noted that such subjects may have normal average daily caloric intake or have dietary behavior characteristic for overnutrition (see e.g., J Gerontol A Biol Sci Med Sci. 2021 Sep. 13; 76(10):1714-1725). Consequently, one or more of the following biomarkers will be followed on an acute (e.g., T0, 30 min, 60 min, 90 min, 120 min, 180 min, 240 min) and long-term (e.g., T0, 1 day, 3 day, 7 day, 14 day, 21 day, 30 day) basis: fasting/average, post-prandial blood glucose and insulin levels, HbA1c, HOMA-IR, triglycerides, total/HDL/oxLDL/LDL cholesterol, lipid particle size, homocysteine, FGF21 expression, SIRT1 expression, body weight, BMI, visceral fat, serum levels of ALT, AST, GST, BUN, creatinine, ketones, and blood pressure. In view of the above, administration of contemplated compositions is therefore expected to positively affect at least one of the listed markers.

Gut microbiome: The effect of contemplated compounds can be evaluated in vitro using the Simulator of the Human Intestinal Microbial Ecosystem (SHIME: see e.g., Chapter 27 in “The Impact of Food Bioactives on Health: in vitro and ex vivo models” by Verhoeckx K, Cotter P, López-Expósito I, et al., editors. Cham (CH): Springer; 2015). Such in vitro model advantageously reduces the impact of other physiological processes that might interfere with the effect of the polyamines on the microbial flora. However, where observations for in vivo conditions are preferred, it should be appreciated that the microbiome can be followed using fecal sampling and 16S rRNA sequencing as is well known in the art. Therefore, microbiome testing will reveal shifts in microbial species/families/orders (e.g., increase in abundance of Firmicutes decrease in abundance of Bacteroides) as well as a change in microbial overall diversity. Moreover, additional parameters that can be obtained from such in vivo and in vitro testing especially include quantification of short chain fatty acids (SCFA), butyrate-producing or bifidobacteria-producing bacteria, and ketone bodies such as (hydroxy)butyric acid, acetate, acetoacetate, propionate in the gut. In view of the above, administration of contemplated compositions is therefore expected to positively affect at least one of the listed markers.

Autophagy: Autophagy and autophagy flux can be measured in cells and in vivo in a number of manners and especially contemplated analyses include detection of autophagic flux by immunoblotting using LC3-II and p62 as indicators, LC3 puncta determination by fluorescence microscopy, measuring degradation of long-lived proteins by the lysosome, GFP-LC3 degradation determined by flow cytometry, LC3-II flux by immunoblotting, accumulation of p62 is tissues, animal models that express mitophagy reporter proteins, etc., and exemplary protocols and references can be found in Int. J. Mol. Sci. 2017, 18, 1865. Additionally, autophagy has also been associated with cardioprotective effects (see e.g., Circ Res. 2017 Apr 14; 120(8): 1229-1231). In view of the above, administration of contemplated compositions is therefore expected to positively affect at least one of the listed markers.

Longevity: It has recently been shown that administration of spermidine, whose intracellular concentration declines during human ageing, markedly extended the lifespan of yeast, flies, worms, and human immune cells. In addition, spermidine administration potently inhibited oxidative stress in ageing mice. Consequently, numerous in vivo model systems (and particularly C. elegans, D. melanogaster, S. cerevisiae, etc.) can be used to determine the effect of polyamines on the model animal. In ageing yeast, spermidine treatment has been shown to trigger epigenetic deacetylation of histone H3 through inhibition of histone acetyltransferases (HAT), to suppress oxidative stress and necrosis. The altered acetylation status of the chromatin led to significant upregulation of various autophagy-related transcripts, triggering autophagy in yeast, flies, worms, and human cells (see e.g., Nat Cell Biol. 2009 November; 11(11):1305-14). In view of the above, administration of contemplated compositions is therefore expected to positively affect at least one of the listed markers.

The aging processes can also be quantitatively followed by measurements of various markers that are often associated with caloric restriction, and especially contemplated markers include Sirtuin 1 activation, inhibition of insulin/insulin growth factor signaling, etc. (see e.g., Cell. 2011 Sep. 2; 146(5):682-95). In view of the above, administration of contemplated compositions is therefore expected to positively affect at least one of the listed markers.

Cognitive and/or memory function: There are numerous cognitive tests known in the art to evaluate cognitive function, execute function, memory function, recall function, etc., which are typically performed in a questionnaire for (on paper or on line). However, further known methods of quantification and/or function of cognitive processes include fMRI and BOLD-fMRI. See also Cell Reports 35, 108985). Improvements in cognition may possibly be due to elevated mitochondrial function in neuronal tissue, which depends on functional autophagy (Atg7) and mitophagy (Pink1), which can be quantified as well to assess changes in cognitive function. In view of the above, administration of contemplated compositions is therefore expected to positively affect at least one of the listed markers.

Gut barrier function: More recent research suggests that various conditions may lead to a partial loss or decline in the intestinal barrier function with attendant leakage of microbial cells and/or cell components into the blood stream. As such, low grade and/or chronic subacute inflammation may develop with a variety of undesirable sequelae. Consequently, improvement in gut barrier function can be measured by reduced endotoxemia (see e.g., J Gerontol A Biol Sci Med Sci. 2021 Sep. 13; 76(10):1714-1725.) Additionally, or alternatively, barrier function can also be assessed via qPCR quantifying mRNA expression of autophagy related genes (e.g., Lc3b, Atg4d, and Atg1612), tight junctions related genes (e.g., Cldn1, Cldn7, Tjp1, and Tjp3), mucin secretion related genes (e.g., Reg3b, Defa, Muc1, and Muc2), and/or inflammation related genes (e.g., Tnfrsf, Tnf-a, and Il6st). In view of the above, administration of contemplated compositions is therefore expected to positively affect at least one of the listed markers.

Dermal structure and skin barrier function: Dermal structure and skin barrier function has been shown to be affected by spermidine, presumably via modulation of the skin microbiome, and especially via Streptococcus abundance. Spermidine was also effective to induce the expression of genes associated with the formation of skin structure and the skin barrier function in human skin cells and improves skin phenotypes such as elasticity, hydration, and desquamation. As such, such gene expression can be quantified for evaluation of the (synergistic) effects of polyamine (see e.g., Commun Biol. 2021 Feb. 19; 4(1):231). In still further aspects, effects of polyamines can be monitored by modulation of human epithelial stem cells (see e.g., PLoS One. 2011; 6(7): e22564). In view of the above, administration of contemplated compositions is therefore expected to positively affect at least one of the listed markers.

In some embodiments, the numbers expressing quantities of ingredients, properties such as concentration, reaction conditions, and so forth, used to describe and claim certain embodiments of the invention are to be understood as being modified in some instances by the term “about.” As used herein, the terms “about” and “approximately”, when referring to a specified, measurable value (such as a parameter, an amount, a temporal duration, and the like), is meant to encompass the specified value and variations of and from the specified value, such as variations of +/−10% or less, alternatively +/−5% or less, alternatively +/−1% or less, alternatively +/−0.1% or less of and from the specified value, insofar as such variations are appropriate to perform in the disclosed embodiments. Thus, the value to which the modifier “about” or “approximately” refers is itself also specifically disclosed. The recitation of ranges of values herein is merely intended to serve as a shorthand method of referring individually to each separate value falling within the range. Unless otherwise indicated herein, each individual value is incorporated into the specification as if it were individually recited herein.

As used herein, the term “administering” a pharmaceutical composition or drug refers to both direct and indirect administration of the pharmaceutical composition or drug, wherein direct administration of the pharmaceutical composition or drug is typically performed by a health care professional (e.g., physician, nurse, etc.), and wherein indirect administration includes a step of providing or making available the pharmaceutical composition or drug to the health care professional for direct administration (e.g., via injection, infusion, oral delivery, topical delivery, etc.). It should further be noted that the terms “prognosing” or “predicting” a condition, a susceptibility for development of a disease, or a response to an intended treatment is meant to cover the act of predicting or the prediction (but not treatment or diagnosis of) the condition, susceptibility and/or response, including the rate of progression, improvement, and/or duration of the condition in a subject.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. As also used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification or claims refer to at least one of something selected from the group consisting of A, B, C . . . and N, the text should be interpreted as requiring only one element from the group, not A plus N, or B plus N, etc.

What is claimed is:

1. A nutritional composition, comprising:
a synergistic combination of a first and a second polyamine in admixture with a nutritionally acceptable carrier;
wherein the first and second polyamine are present in quantities that synergistically increase a healthspan of a subject; and
wherein the composition is formulated for oral consumption or topical application.

2. The composition of claim 1, wherein the first and second polyamine are selected from the group consisting of spermidine, spermine, putrescine, cadaverine, agmatine, and ornithine.

3. The composition of claim 2, wherein the first and/or second polyamine are present in the composition as a free base or as a hydrochloride salt or from a natural source.

4. The composition of claim 1, wherein a dosage unit of the composition contains spermidine in an amount of at least 0.1 mg, spermine in an amount of at least 0.05 mg, putrescine in an amount of at least 0.01 mg, cadaverine in an amount of at least 0.1 mcg, agmatine in an amount of at least 1 mg, and/or L-ornithine in an amount of at least 0.01 mg.

5. The composition of claim 4, wherein the dosage unit of the composition contains spermidine in an amount of equal or less than 50 mg, spermine in an amount of equal or less than 20 mg, putrescine in an amount of equal or less than 5 mg, cadaverine in an amount of equal or less than 50 mcg, agmatine in an amount of equal or less than 1,000 mg, and/or L-ornithine in an amount of equal or less than 1,000 mg.

6. The composition of claim 5, wherein the dosage unit comprises equal or less than 1,000 mg of the composition, and wherein the first and second polyamines account for at least 20 wt % of the composition.

7. The composition of claim 1, wherein the first and second polyamine are present in synergistic quantities with respect to
(a) increase the healthspan of a subject by increasing locomotor activity;
(b) increase the healthspan of a subject by preserving or increasing the health of thin or thick filaments of cellular muscle fibers;
(c) increase the healthspan of a subject by preserving or increasing the stimulation of regions of the brain associated with reward or happiness;
(d) increase the healthspan of a subject by decreasing concentrations of cortisol in the nervous system;
(e) increase the healthspan of a subject by preserving or increasing the energy yield of metabolic processes;
(f) increase the healthspan of a subject by preserving or increasing the health of extracellular matrices;
(g) increase the healthspan of a subject by preserving or increasing intracellular NAD+or ATP concentration;
(h) increase the healthspan of a subject by preserving or increasing mitochondrial biogenesis; and/or
(i) increase the healthspan of a subject by preserving or increasing glucose uptake into cells.

8. The composition of claim 1, further comprising at least one additional agent that supports metabolism.

9. The composition of claim 8, wherein the additional agent that supports metabolism comprises fish oil, melatonin, capsaicin, theacrine, methylliberine, berberine, vitamins, healthy acids, turmeric, caffeine, green tea, protein, or fiber.

10. The composition of claim 1, wherein the composition is formulated as a capsule, a tablet, a gummy, dissolvable strip, a gel, a powder, or a drink.

11. The composition of claim 1, wherein the composition is formulated as a cream, an ointment, a lotion, a serum, a soaps, a shampoo, or a conditioner.

12. A method of modulating a healthspan of a subject, comprising:
orally or topically administering a nutritional composition comprising a synergistic combination of a first and a second polyamine with a nutritionally acceptable carrier;
wherein the first and second polyamine are present in quantities that synergistically increase the healthspan of a subject.

13. The method of claim 12, wherein the first and second polyamine are selected from the group consisting of spermidine, spermine, putrescine, cadaverine, agmatine, and ornithine, and wherein the first and/or second polyamine are present in the composition as a free base or as a hydrochloride salt or from a natural source.

14. The method of claim 12, wherein a dosage unit of the composition contains spermidine in an amount of between 0.1 mg and 50 mg, spermine in an amount of between 0.05 mg and 20 mg, putrescine in an amount of between 0.01 mg and 5 mg, cadaverine in an amount of between 0.1 mcg and 50 mcg, agmatine in an amount of between 1 mg and 1,000 mg, and/or L-ornithine in an amount of between 0.01 mg and 1 g.

15. The method of claim 12, wherein the method of modulating the healthspan preserves or increases locomotor activity, health of thin or thick filaments of cellular muscle fibers, stimulation of brain regions associated with reward or happiness, energy yield of metabolic processes, health of extracellular matrices, intracellular concentration of NAD⁺ or ATP, mitochondrial biogenesis, and/or glucose uptake into cells, and/or decreases concentration of cortisol in the nervous system.

16. The method of claim 12, wherein the nutritional composition further comprises fish oil, melatonin, vitamins, capsaicin, theacrine, methylliberine, berberine, healthy acids, turmeric, caffeine, green tea, protein, and/or fiber.

17. A method of manufacturing a nutritional composition, comprising:
compounding a first and a second polyamine with a nutritionally acceptable carrier, and formulating the first and the second polyamine and the carrier into an oral or topical formulation;
wherein the first and second polyamine are present in the formulation in quantities that, upon administration to a subject, synergistically increase a healthspan of the subject.

18. The method of claim 17, wherein a dosage unit of the composition contains spermidine in an amount of at least 0.1 mg, spermine in an amount of at least 0.05 mg, putrescine in an amount of at least 0.01 mg, cadaverine in an amount of at least 0.1 mcg, agmatine in an amount of at least 1 mg, and/or L-ornithine in an amount of at least 0.01 mg.

19. The method of claim 18, wherein a dosage unit of the composition contains spermidine in an amount of equal or less than 50 mg, spermine in an amount of equal or less than 20 mg, putrescine in an amount of equal or less than 5 mg, cadaverine in an amount of equal or less than 50 mcg, agmatine in an amount of equal or less than 1,000 mg, and/or L-ornithine in an amount of equal or less than 1,000 mg.

20. The method of claim 17 wherein the oral formulation is a powdered formulation, or wherein the oral formulation is encapsulated or compressed to form a tablet or to form a single-dosage unit, or wherein the oral formulation is a ready-to-use beverage.
